(19) United States
(12) Patent Application Publication
(10) Pub. No.: US 2009/0030260 A1
(43) Pub. Date: Jan. 29, 2009

(54) SEED ANCHOR

(75) Inventor: Felix W. MICK, Bronxville, NY (US)

Correspondence Address: KENYON & KENYON LLP, ONE BROADWAY, NEW YORK, NY, 10004, US

(73) Assignee: Mick Radio-Nuclear Instruments, Inc., Mt. Vernon, NY (US)

(21) Appl. No.: 12/180,120

(22) Filed: Jul. 25, 2008

Related U.S. Application Data
(60) Provisional application No. 60/951,786, filed on Jul. 25, 2007.
(60) Provisional application No. 61/037,264, filed on Mar. 17, 2008.

(51) Int. Cl. A61M 36/12

(52) U.S. Cl. 600/7

(57) ABSTRACT

A seed anchor includes a tubular anchor body having a plurality of partial cylindrical walls splayed outwardly at an axial end of the anchor body to form an anchoring structure configured to resist axial movement of the seed anchor in a direction opposite an insertion direction when the seed anchor is implanted into tissue.

### RELATED APPLICATIONS

This application claims benefit to U.S. Provisional Application No. 60/951,786, filed on Jul. 25, 2007, and U.S. Provisional Application No. 61/037,264, filed on Mar. 17, 2008, each of which is expressly incorporated herein in its entirety by reference thereto.

### FIELD OF THE INVENTION

The present invention relates generally to brachytherapy. More specifically, the present invention relates to a device and method for placement of radioactive therapeutics to the tissue of a patient.

### BACKGROUND INFORMATION

Brachytherapy is a form of cancer treatment in which radiation sources are placed inside a patient's body to irradiate a tumor. In brachytherapy, a physician typically implants several radioactive seeds in or around a tumor, thereby providing a higher radiation dose to the tumor than would be possible with external beam radiation therapy. Careful and precise placement of the radioactive seeds allows for localized and precise irradiation of the tumor.

The radioactive seeds are introduced to the tissue via an insertion device or applicator, typically a needle or similar device. The insertion device may have a plurality of radioactive seeds, and, optionally, spacers therebetween to ensure proper spacing within the tissue. The seeds may be preloaded in a cartridge.

A problem with the aforementioned treatment method is that the seeds can shift or migrate once they are inserted in the tissue. This problem can arise, e.g., when the insertion device is being removed from the patient, e.g., due to a suction effect during withdrawal of the applicator needle. When the insertion device is inserted, it pierces the tissue, creating a longitudinal opening. The seeds can later shift along this opening, deviating from their intended, efficacious positions. In some cases, these seeds can migrate to unintended areas, such as the lungs or bloodstream. While stranded seeds (in which the seeds each have a hole through which a common thread is inserted) can lower the potential for seed migration, problems persist. For example, as the insertion device is removed from the opening, a negative pressure can be created at a trailing edge. This negative pressure can cause displacement of the seeds in the removal direction along the longitudinal opening, even if the seeds are stranded. Even if the removal of the insertion device does not cause displacement, the seeds can shift as a result of other factors, e.g., motion of the patient and/or gravity. Regardless of the cause, this shifting may have a detrimental effect on the critical placement of the radioactive seeds.

### SUMMARY

Example embodiments of the present invention provide a seed anchor for reducing, minimizing or even preventing the displacement of radioactive seeds after insertion into a patient's tissue. The seed anchor serves to properly space the radioactive seeds during insertion and to anchor the seeds after insertion into the tissue. The seed anchor may be self-actuating so that, upon insertion, the anchor expands to engage the surrounding tissue. The expansion may be actuated by spring loading or biasing of at least one spring finger and/or the expansion may be actuated by a radially compressed coil. The seed anchor may have at least one pin that extends axially with respect to the seed. The seed anchor may be attached to or separate from the radioactive seeds. The seed anchor may be made from a single piece to reduce manufacturing costs. The seed anchor may be formed from titanium, stainless steel, absorbable suture material, synthetic suture material, or any other material approved for permanent seed implants. The seed anchor may be formed of a shape memory alloy, e.g., a nickel-titanium alloy, Nitinol, etc.

An anchoring mechanism may have a self-actuating seed anchor that expands during insertion to press against surrounding tissue and to prevent movement and/or rotation of the radioactive seed. The seed anchor may be preloaded into a seed cartridge or into an applicator needle to efficaciously space apart sequential radioactive seeds during insertion.

According to an example embodiment of the present invention, a seed anchor includes a hollow tubular anchor body having a first axial end, a second axial end, and a plurality of notches extending from the first axial end to a point between the first axial end and the second axial end to form therebetween a plurality of arms arranged to resist axial movement of the seed anchor when the seed anchor is implanted into tissue.

The arms may be splayed outwardly toward the first end.

The arms may be elastically flexible to allow the arms to be radially compressed into a preloaded position.

The anchor body has a constant diameter along the entire axial length of the anchor body when the arms are in the preloaded position.

The seed anchor may be monolithically formed as a single piece.

The anchor body may be attached to a radioactive seed.

The second axial end of the anchor body may be attached to an end of the radioactive seed, where the plurality of arms extends axially away from the radioactive seed.

The seed anchor may be configured to be loaded into an applicator needle with the radioactive seed.

The arms may be equally spaced around a circumference of the anchor body.

The seed anchor may include six arms.

The seed anchor may be configured to be loaded into an applicator needle between radioactive seeds to axially space apart the radioactive seeds.

The seed anchor may be formed from absorbable suture material.

The seed anchor may be formed from a shape memory alloy.

According to an example embodiment of the present invention, a seed anchor includes an anchor body having a plurality of pins, each pin axially extending to a free end to form an anchoring structure arranged to resist axial movement of the seed anchor when the seed anchor is implanted into tissue.

The plurality of pins may be equally spaced around a circumference of the seed anchor.

Each of the plurality of pins may be arranged to be parallel to an insertion direction when the seed anchor is implanted.

The seed anchor may be attached to an end of a radioactive seed.

According to an example embodiment of the present invention, a seed anchor includes an axially extending coil attached to a radioactive seed, the coil forming an anchoring structure arranged to resist axial movement of the radioactive seed when the radioactive seed is implanted into tissue.

The coil may have a constant diameter along its axial length and/or extend to a free end.

According to an example embodiment of the present invention, a radioactive seed cartridge includes a plurality of radioactive seeds preloaded in a cartridge chamber and a seed anchor preloaded in the cartridge chamber, the seed anchor arranged to resist axial movement of at least one of the radioactive seeds when the seed anchor is implanted into tissue.

The seed anchor may be disposed radially between two of the radioactive seeds.

According to an example embodiment of the present invention, a spacer cartridge may include a plurality of seed anchors preloaded in a chamber. The spacer cartridge may be arranged to dispense anchors radially into a cylinder or applicator needle of a seed applicator.

According to an example embodiment of the present invention, a method of implanting radioactive seeds includes preloading a radioactive seed cartridge with radioactive seeds and a seed anchor in an axial row in a chamber of the cartridge. The method may include pushing the radioactive seeds and the seed anchor out of the cartridge, where the seed anchor resists axial motion of at least one of the radioactive seeds.

The seed anchor may be attached to an end of one of the radioactive seeds.

The seed anchor may expand upon implantation.

Further features and aspects of example embodiments of the present invention are described in more detail below with reference to the appended Figures.

### BRIEF DESCRIPTION OF THE DRAWINGS

FIG. 1 illustrates an applicator device loaded with a series of radioactive seeds and seed anchors.

FIG. 2 illustrates an applicator device, a radioactive seed, and a seed anchor in an open position.

FIG. 3 illustrates an applicator device, a radioactive seed and a seed anchor in both a closed position and an open position.

FIG. 4 is a perspective view of an applicator device, a radioactive seed and a seed anchor in both a closed and an open position.

FIG. 5 illustrates a seed anchor and multiple radioactive seed configurations.

FIG. 6 illustrates a seed anchor and multiple radioactive seed configurations.

FIG. 7 illustrates a radioactive seed with different seed anchor arrangements.

FIG. 8 illustrates a radioactive seed with a coiled seed anchor.

FIG. 9 illustrates a radioactive seed cartridge with a seed anchor.

FIG. 10 illustrates a seed anchor cartridge with seed anchors.

FIG. 11 illustrates a seed anchor cartridge with seed anchors attached to radioactive seeds.

### DETAILED DESCRIPTION

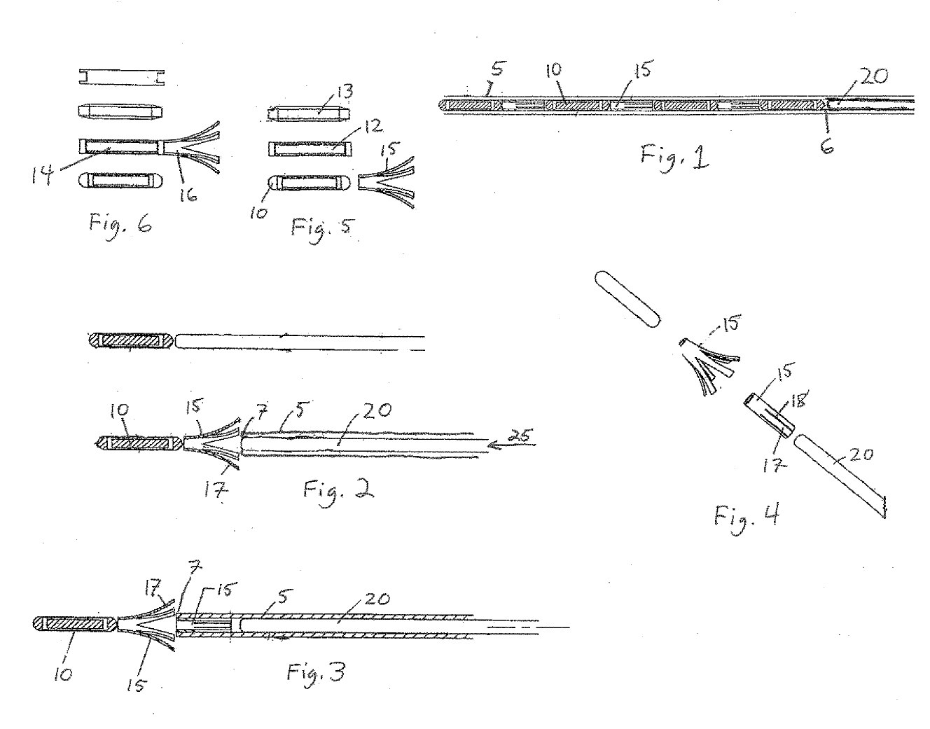

FIG. 1 illustrates an applicator device 5 loaded with a series of radioactive seeds 10 and seed anchors 15. The applicator device 5 has an internal bore 6 into which the radioactive seeds 10 and seed anchors 15 are loaded and in which a plunger device 20, such as, e.g., a pushwire or stylet, is located. The seeds 10 and seed anchors 15 may be preloaded in bore 6 or may be provided in a cartridge. The seed anchors depicted in FIG. 1 are arranged in the bore 6 in a closed position. If provided in a cartridge, the seed anchors 15 may be provided in a closed position or may be provided in an open position and then be urged into a closed position upon insertion into the applicator needle.

The internal bore 6 has a diameter that is approximately the same as the diameters of the radioactive seeds 10 and the seed anchors 15, but is dimensioned to allow the radioactive seeds 10 and seed anchors 15 to slide longitudinally therein. The seeds 10 may have a diameter of, e.g., 0.8 mm and a length of, e.g., 4.5 mm to 5.0 mm. The seed anchor 15 may have a length of, e.g., 3.0 mm. As illustrated in FIG. 1, the seed anchors 15 provide a predetermined spacing between adjacent radioactive seeds 10. While the radioactive seeds 10 and seed anchors 15 alternate sequentially as depicted in FIG. 1, it should be appreciated that other arrangements are possible, such as, e.g., having a seed anchor between every other seed or having multiple seed anchors between seeds. It should be further appreciated that the seed anchors 15 may be loaded in a reverse position to that illustrated in FIG. 1. The seed anchors 15 are shown in a preloaded position.

FIG. 2 illustrates the applicator device 5, a radioactive seed 10, and a seed anchor 15 in an open position. During insertion, the plunger device 20 of the applicator device 5 moves in an insertion direction 25 to expel the radioactive seed 10 and seed anchor 15. When the seed anchor 15 passes from inside the bore to outside the bore, the seed anchor 15 automatically expands from the closed position to the open position. In the open position, a trailing portion of the seed anchor 15 expands radially outwardly into the position illustrated in FIG. 2. The seed anchor 15 reduces the likelihood or even prevents at least rearward motion (i.e., motion in the direction opposite insertion direction 25) as a result of arms or spring fingers 17. The spring fingers 17, as shown in FIG. 2, are in a relaxed state. In order to load the seed anchor 15 into the applicator device 10, the partial cylindrical walls or spring fingers 17 are flexed radially inwardly. This spring loading provides for a seed anchor 15 that is self-actuating. Thus, when the trailing end of the seed anchor 15 clears an opening 7 of the insertion device 5, the arms or spring fingers 17 spring radially outwardly from a preloaded position to an open position. When inserted into tissue, this outward springing biases the spring fingers 17 into the surrounding tissue, providing an anchoring effect. This reduces or even prevents sliding of the seed anchor 15 in at least the direction of the spring fingers, due to both friction and interference between the ends of the spring fingers and the tissue. Although the seed anchor 15 depicted in FIG. 2 has spring fingers that open on a trailing end, it should be appreciated that other arrangements are possible. For example, a seed anchor 15 may have spring fingers on a leading end, in which case the seed anchor 15 would tend to gradually open as the leading end proceeds through the opening 7 of the applicator device 5. A seed anchor 15 may also have spring fingers on both the leading end and the trailing end, thereby preventing motion in both directions. It should be appreciated that other methods of self-actuation may be employed, such as, e.g., compressible rebound materials including certain foams. While six spring fingers 17 are illustrated, e.g., in FIG. 4, any appropriate number of spring fingers 17 may be provided. It should be further appreciated that the arms 17 may not be self-actuating.

After being dispensed from the applicator device 5, the seed anchor 15 reduces or even prevents shifting or migration of the radioactive seed 10 by providing a positive stop. According to the example illustrated in FIG. 2, the positive stop is formed between the leading end of the seed anchor 15 and the trailing end of the radioactive seed 10. Referring to FIGS. 1 and 2, the seed anchor serves a dual function of providing spacing between adjacent radioactive seeds 10 during insertion and anchoring the radioactive seeds after insertion.

FIG. 3 illustrates an applicator device 5, a radioactive seed 10 and a seed anchor 15 in both a closed position and an open position.

FIG. 4 is a perspective view of an applicator device, a radioactive seed 10 and a seed anchor 15 in both a closed and an open position. Although the seed anchor 15 depicted in FIG. 3 is illustrated as hollow and having six spring fingers, it should be appreciated that the seed anchor 15 may be solid and/or have any number of spring fingers. The seed anchor 15 is formed from a hollow tube or cylinder with notches or slots 18 on one end to form partial cylindrical walls or spring fingers 17 therebetween that axially extend from the tubular or cylindrical seed anchor body and splay outwardly in a radial direction. In this regard, the seed anchor 15 may be formed monolithically from a single piece of material. It should be appreciated, however, that other examples may be formed from separate pieces. For example, separate spring fingers may be joined, e.g., hingedly, to the seed anchor 15. The seed anchor 15 may be formed from titanium, stainless steel, absorbable suture material, synthetic suture material, or any other material approved for permanent seed implants. The material may optionally be textured to improve friction with the tissue.

FIG. 5 illustrates a seed anchor 15 and various radioactive seed configurations. The seed anchor 15 effectively anchors seeds 10, 12, 13, as well seeds having other configurations. As illustrated in FIG. 5, the seed anchor 15 and seed 10 may be separate structures.

FIG. 6 illustrates a seed anchor 16 and various radioactive seed configurations. The seed anchor 16 is fixedly attached to the radioactive seed 14. The seed anchor 16 can be integrally formed with the radioactive seed 14, or it may be formed separately and subsequently attached to the seed 14. It may be beneficial to have the seed anchor 16 attached to the radioactive seed 14 because, e.g., it would eliminate the possibility of the seed slipping past the adjacent end of the seed anchor and can prevent rotation of the seed along its longitudinal axis. Like the example seed anchor 15 of FIGS. 1 to 5, the seed anchor 16 both provides spacing between adjacent seeds during insertion and anchors the seeds after insertion. The seed anchor 16 may have a diameter that is identical to the diameter of the radioactive seed 14 at the interface between the seed and the seed anchor. However, the diameter of the seed anchor 16 may be greater or less than that of the radioactive seed 14. Although the seed anchor 16 and the radioactive seed 16 are fixedly attached as depicted in FIG. 6, it should be appreciated that, according to other examples, the seed anchor may be rotatably attached or attached by alternative methods, e.g., a tether. Similar to the example seed anchor 15 of FIGS. 1 to 5, the seed anchor 16 both provides spacing between adjacent seeds during insertion and anchors the seeds after insertion.

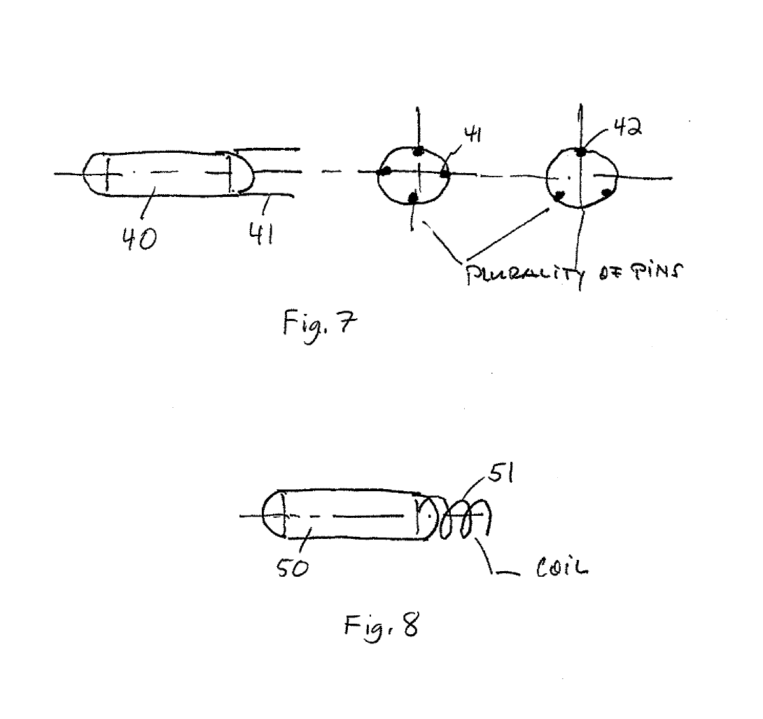

FIG. 7 illustrates a radioactive seed 40 with different seed anchor arrangements. The seed anchor is provided in the form of a plurality of pins 41, 42 that extend in an axial direction away from the radioactive seed 41. In one arrangement, the anchor has four pins 41, while in another arrangement, the anchor has three pins 42. However, it should be appreciated that any number of pins, including a single pin, may be employed. It should also be appreciated that although the pins illustrated in FIG. 7 only extend from one end of the seed, according to other examples, pins may extend from both ends of the seed. In the arrangement shown in FIG. 7, the free ends of the pins may engage surrounding tissue when inserted into a patient's body to prevent movement of the radioactive seed 40. Although the pins 41, 42 shown in FIG. 7 are straight and parallel to the longitudinal axis of the seed 40 and located at the outer periphery of the cross section of the seed 40, it should be appreciated that, according to other examples, other arrangements may be used. For example, the pins could be positioned so as to be located radially closer to the longitudinal axis and/or may be angled outwardly or inwardly as the pins extend axially away from the seed. The pins may also be non-linear, e.g., curved. The pins may also be arranged so as to spring radially outwardly so as to self-actuate upon insertion. The free ends of the pins 41, 42 may be blunt, e.g., rounded or squared, or sharp. While the pins 41, 42 are illustrated as being symmetrically arranged, it should be appreciated that asymmetrical arrangements of pins 41, 42 may be provided.

FIG. 8 illustrates a radioactive seed 50 with a coiled seed anchor 51. When inserted into a patient's body the coil may prevent movement of the seed 50 because of friction and/or interference with tissue that extends into the space between adjacent windings of the coil 51. The coil may have a constant coil radius or a coil radius that varies along the axial length of the coil. Moreover, it should be appreciated that the coil may be arranged so that, prior to insertion, the coil has a compressed diameter. In this manner, the coil may self-actuate upon insertion, as the diameter expands outwardly. This may result in a more secure anchoring, as the coil presses into the surrounding tissue. Although the coil shown in FIG. 8 is a single helical coil, it should be appreciated that, according to other examples, multiple helices may be employed, e.g., a double helix. It should be further appreciated that the coil may be wound at an angle that varies along the axial length of the coil such that, e.g., the spacing between adjacent windings varies.

The seed anchors 41, 42, 51 shown in FIGS. 7 or 8 may be attached to the seeds 40, 50 or may be separate parts. In addition to anchoring the seeds 40, 50, these anchors, whether attached or unattached, may further serve to space apart axially adjacent seeds. As with the other embodiments described herein, the seed anchors 41, 42, 51 may be formed from any appropriate material, e.g., titanium, stainless steel, absorbable suture material, synthetic suture material, or any other material approved for permanent seed implants. The seed anchors 41, 42, 51 may be formed of a shape memory alloy, e.g., a nickel-titanium alloy, Nitinol, etc.

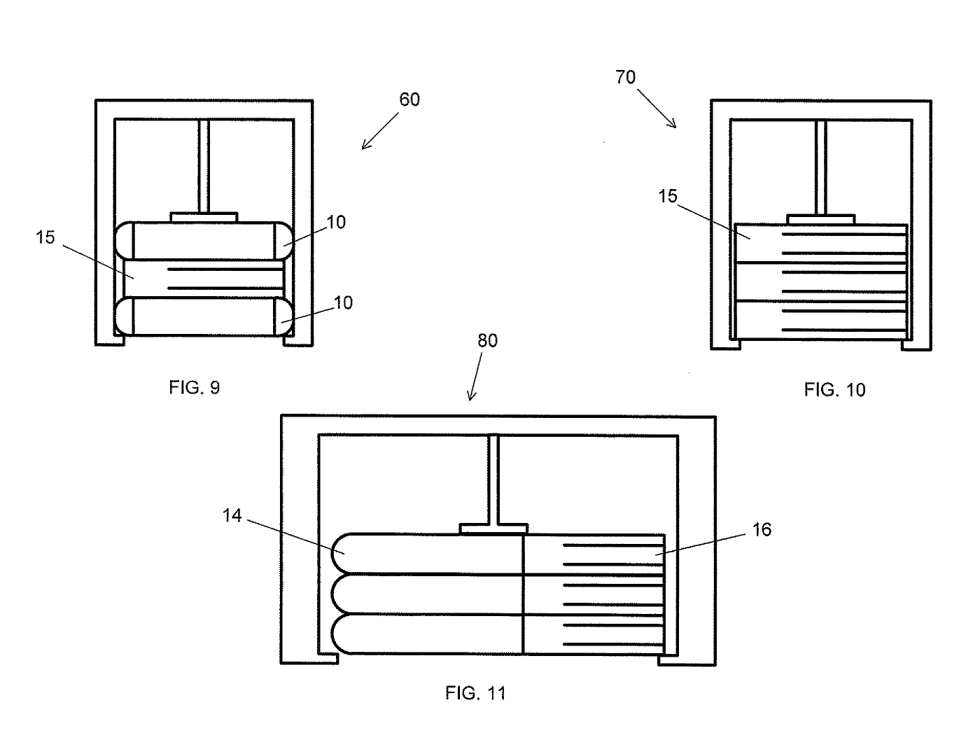

FIG. 9 schematically illustrates a radioactive seed cartridge 60 with a seed anchor 15, while FIG. 10 schematically illustrates a seed anchor cartridge 70 with seed anchors 15. The cartridge 60 and or the cartridge 70 may be used to load radioactive seeds 10 and/or seed anchors into the applicator device 10, shown in FIGS. 1 to 3. The seed cartridge 60 includes both radioactive seeds 10 and seed anchors 15, where the seeds 10 and anchors 15 are separate and sequenced such that as the seeds 10 and anchors 15 are loaded, e.g., in a radial direction, into the applicator device 10 in a particular order. The order may be adjusted depending on, e.g., the nature of the treatment. Cartridges 60 may be preloaded in particular sequences to suit different needs.

Cartridge 70 differs in that it only provides seed anchors 15. Cartridge 70 may, e.g., be used in conjunction with another cartridge having only radioactive seeds, such that the cartridges are selected to dispense anchors and seeds, respectively, in a particular order, e.g., alternating between one seed and one anchor such that a single anchor spaces apart each seed.

FIG. 11 illustrates a cartridge 80 that includes radioactive seeds 14 attached to seed anchors 15. In this regard, the seed/anchor units 14, 15, may be provided, e.g., in a radial direction, to the applicator device 10.

While the cartridges 60, 70, 80 illustrate anchors 15 and 16, it should be appreciated that the cartridges 60, 70, 80 may include any other anchor described herein. Moreover, and of the cartridges may house any combination of seeds and anchors in any orientation, e.g., with some anchors having arms that extend in the opposite axial direction as some other anchors of the same cartridge.

It should be appreciated that the features of aforementioned examples and embodiments may be combined with other, e.g., conventional, anti-migration mechanisms. For example, the seeds and/or seed anchors may be stranded by, e.g., absorbable suture material.

Although the present invention has been described with reference to particular examples and embodiments, it should be understood that the present invention is not limited to those examples and embodiments. Moreover, the features of the particular examples and embodiments may be used in any combination. The present invention therefore includes variations from the various examples and embodiments described herein, as will be apparent to one of skill in the art.

What is claimed is:

1. A seed anchor, comprising:
a hollow tubular anchor body having
a first axial end,
a second axial end, and
a plurality of notches extending from the first axial end to a point between the first axial end and the second axial end to form therebetween a plurality of arms configured to resist axial movement of the seed anchor when the seed anchor is implanted into tissue.

2. The seed anchor according to claim 1, wherein the arms are splayed outwardly toward the first end.

3. The seed anchor according to claim 2, wherein the arms are elastically flexible to allow the arms to be radially compressed into a preloaded position.

4. The seed anchor according to claim 3, wherein the anchor body has a constant diameter along the entire axial length of the anchor body when the arms are in the preloaded position.

5. The seed anchor according to claim 1, wherein the seed anchor is monolithically formed as a single piece.

6. The seed anchor according to claim 1, wherein the anchor body is attached to a radioactive seed.

7. The seed anchor according to claim 6, wherein the second axial end of the anchor body is attached to an end of the radioactive seed, wherein the plurality of arms extends axially away from the radioactive seed.

8. The seed anchor according to claim 7, wherein the seed anchor is configured to be loaded into an applicator needle with the radioactive seed.

9. The seed anchor according to claim 1, wherein the arms are equally spaced around a circumference of the anchor body.

10. The seed anchor according to claim 1, wherein the seed anchor includes six arms.

11. The seed anchor according to claim 1, wherein the seed anchor is configured to be loaded into an applicator needle between radioactive seeds to axially space apart the radioactive seeds.

12. The seed anchor according to claim 1, wherein the seed anchor is formed from absorbable suture material.

13. The seed anchor according to claim 1, wherein the seed anchor is formed from a shape memory alloy.

14. A seed anchor, comprising:
an anchor body having a plurality of pins, each pin extending axially distally from an axial distal end of the anchor body to a free end to form an anchoring structure configured to resist axial movement of the seed anchor when the seed anchor is implanted into tissue.

15. The seed anchor according to claim 14, wherein the plurality of pins is equally spaced around a circumference of the seed anchor.

16. The seed anchor according to claim 14, wherein each of the plurality of pins is arranged to be parallel to an insertion direction when the seed anchor is implanted.

17. The seed anchor according claim 14, wherein the seed anchor is attached to an end of a radioactive seed.

18. A seed anchor, comprising:
an axially extending coil attached to a radioactive seed, the coil forming an anchoring structure configured to resist axial movement of the radioactive seed when the radioactive seed is implanted into tissue.

19. The seed anchor according to claim 18, wherein the coil has a constant diameter along its axial length.

20. The seed anchor according to claim 18, wherein the coil extends to a free end.
